(19) United States
(12) Patent Application Publication
(10) Pub. No.: US 2024/0359536 A1
(43) Pub. Date: Oct. 31, 2024

(54) Replaceable Cartridge Having at Least One Scent Carrier

(71) Applicant: Bayerische Motoren Werke Aktiengesellschaft, Muenchen (DE)

(72) Inventor: Etienne ILIFFE-MOON, Menlo Park, CA (US)

(21) Appl. No.: 18/141,030

(22) Filed: Apr. 28, 2023

(51) Int. Cl. B60H 3/00

(52) U.S. Cl. CPC B60H 3/0007; B60H 2003/0057

(57) ABSTRACT

A replaceable cartridge for a scent dispensing system includes a scent medium having at least one scent carrier and a body surrounding a chamber for housing the scent medium. A surface area of the scent medium is configured to control a diffusion rate of a scent from the scent medium, and a shape of the scent medium remains constant in different environmental conditions. At least a portion of the body is transparent or translucent.

### BACKGROUND AND SUMMARY OF THE INVENTION

The present invention relates to a replaceable cartridge having at least one scent carrier, as well as a scent dispensing system including the replaceable cartridge and a vehicle including the scent dispensing system. The present invention also relates to a method of forming the replaceable cartridge.

Many vehicles include fragrances to enhance the experience of the driver and passengers in the vehicle. Liquids, such as solvent-based liquids or water-based liquids, have traditionally been used as carriers for fragrances. However, liquid fragrances should be avoided in vehicles due to environmental factors such as the temperature and humidity, which can cause off-gassing, loss of chemical stability, and degradation.

The packaging of fragrances is important to the consumer experience and the consumer perception of the product. For example, the perfumery industry uses elaborate packages that may include expensive glass bottles, caps, and external boxes. In addition, the perfumery industry has marketed the fragrances by telling a story to engage the consumer.

The consumer packaged goods market uses dry-scent media that packages fragrances in polymer beads. However, these beads are chopped strands of polymer that do not look attractive, and do not provide a suitable appearance for a premium fragrance. In addition, the beads are not designed to provide a desired diffusion rate of the scent.

Exemplary embodiments of the invention encapsulate a scent in a polymer substrate, such as polyethylene or thermoplastic, by performing injection molding or extrusion molding. The surface area of the scent medium is formed to control the diffusion rate of the scent from the scent medium. For example, the shape of the scent medium may be chosen to provide the desired diffusion rate.

In addition, various characteristics of the scent medium, such as the color, texture, gloss, and shape, may be chosen to optimize the appearance of the scent medium. Research studies have shown that color influences a person's perception of scent. Other physical attributes, such as the texture, gloss, and shape, may also influence the person's perception of the scent beyond olfaction alone. The scent medium is enclosed within a cartridge body, at least a portion of which is transparent or translucent in order to display the scent medium. At a point of purchase, the scent may be displayed so that the consumer may see and smell the fragrances. Additionally, the scent may be packaged as a promotion, such as a mailed promotion or marketing campaign, or used in an additional ambient diffuser product for home use. For example, the in-vehicle cartridge may be packaged with an additional ambient diffuser for use at home, which also may be packaged in a clear material.

According to an aspect of the invention, a replaceable cartridge for a scent dispensing system is provided. The replaceable cartridge includes a scent medium having at least one scent carrier and a body surrounding a chamber for housing the scent medium. A surface area of the scent medium is configured to control a diffusion rate of a scent from the scent medium, and a shape of the scent medium remains constant in different environmental conditions. At least a portion of the body is transparent or translucent.

The at least one scent carrier may have a hollow shape or an open shape. Alternatively or additionally, the at least one scent carrier may have an open cavity. Alternatively or additionally, the at least one scent carrier may have a closed shape.

The at least one scent carrier may include a plurality of scent carriers. The scent medium may include air cavities between adjacent ones of the plurality of scent carriers. Alternatively or additionally, some of the plurality of scent carriers may have interlocking shapes. Alternatively or additionally, respective surface areas of the plurality of scent carriers may be configured to control the diffusion rate of the scent from the scent medium.

A color, a texture, a gloss, and/or the shape of the scent medium may be configured to correspond to the scent. The scent medium and/or the body may include a compostable material.

The body may include an aperture that enables air to flow through the chamber. The replaceable cartridge may also include a valve that is arranged at the aperture.

According to another aspect of the invention, a scent dispensing system is provided. The scent dispensing system includes the replaceable cartridge, an airflow source that is configured to generate an airflow, and a controller that is configured to control the airflow source and the valve to send the airflow through the chamber.

According to another aspect of the invention, a vehicle is provided. The vehicle includes the scent dispensing system, which is integrated into a heating, ventilation, and air conditioning system of the vehicle. The vehicle also includes a user interface that is configured to display a visual representation of the scent medium.

According to another aspect of the invention, a method of forming the replaceable cartridge is provided. The method includes encapsulating a scent in a polymer material, performing injection molding or extrusion molding of the polymer material to form a scent medium comprising at least one scent carrier, and enclosing the scent medium within a chamber surrounded by a body. A surface area of the scent medium is formed to control a diffusion rate of a scent from the scent medium, and a shape of the scent medium is formed to remain constant in different environmental conditions.

Other objects, advantages and novel features of the present invention will become apparent from the following detailed description of one or more preferred embodiments when considered in conjunction with the accompanying drawings.

### BRIEF DESCRIPTION OF THE DRAWINGS

FIG. 1 shows a cross-sectional view of a scent dispensing system according to an exemplary embodiment of the invention.

FIGS. 2A-2D show examples of scent carriers having hollow or open shapes.

FIGS. 3A-3C show examples of scent carriers having open cavities.

FIGS. 4A-4C show the corresponding cross-sections of the scent carriers shown in FIGS. 3A-3C.

FIGS. 5A-5E show examples of scent carriers having closed shapes.

FIG. 6 shows an example in which the scent medium includes a plurality of self-organizing scent carriers.

FIGS. 7A-7C show examples of scent carriers that may be used as the self-organizing scent carriers.

FIGS. 8A and 8B show examples in which the scent medium includes a plurality of interlocking scent carriers.

FIGS. 9A and 9B show additional examples in which the scent medium includes a plurality of interlocking scent carriers.

FIGS. 10A and 10B show examples of replaceable cartridges in which the scent medium includes a plurality of scent carriers.

FIG. 11 shows an example of a replaceable cartridge in which the scent medium includes a plurality of scent carriers having different shapes.

FIG. 12 shows a flowchart of a method of forming a replaceable cartridge for a scent dispensing system according to an exemplary embodiment of the invention.

### DETAILED DESCRIPTION OF THE DRAWINGS

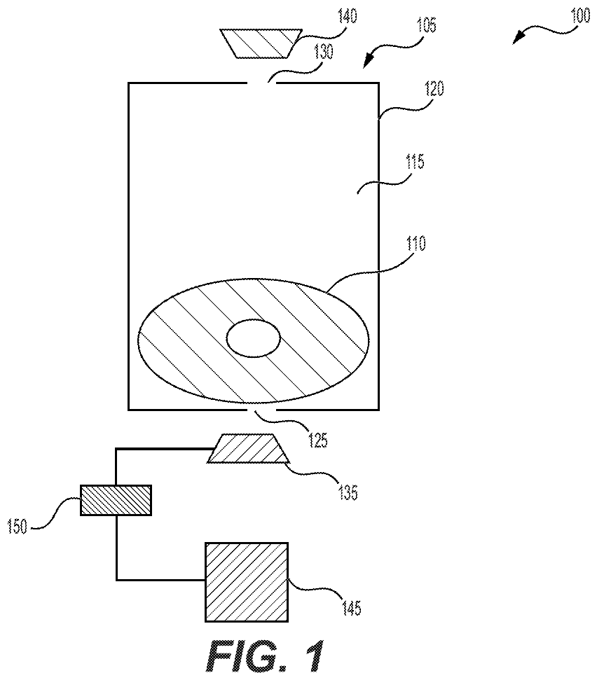

FIG. 1 shows a cross-sectional view of a scent dispensing system 100 according to an exemplary embodiment of the invention. As shown in FIG. 1, the scent dispensing system 100 includes a replaceable cartridge 105, which has a body 120 that surrounds a chamber 115. A scent medium 110 is contained within the chamber 115. At least a portion of the body 120 is transparent or translucent in order to display the scent medium 110. For example, the body 120 may be entirely transparent or translucent, or may include a window or windows that are transparent or translucent. The body 120 may include various materials, such as glass, polymer, silicone, metal, etc.

In order to allow air to flow in and out of the body 120, the body 120 may include a single aperture or a plurality of apertures, such as a first aperture 125 and a second aperture 130. A first valve 135 may be arranged at the first aperture 125, and a second valve 140 may be arranged at the second aperture 130. The first valve 135 and the second valve 140 may be integrated with the replaceable cartridge 105, such that they are replaced along with the replaceable cartridge 105. Alternatively, the first valve 135 and the second valve 140 may be separate from the replaceable cartridge 105 and instead attached to a component within the vehicle.

An airflow source 145 may be configured to generate an airflow. A controller 150 may be provided to control the airflow source 145, the first valve 135, and the second valve 140 to send the airflow through the chamber 115. The controller 150 may receive information about the configuration of the replaceable cartridge 105 in order to automatically calibrate the scent dispensing system 100 for different cartridges. The information may include details about the scent medium 110, such as the size, shape, format, scent, scent substrate, etc. The information may be automatically communicated by the scent dispensing system 100 or manually communicated by the user, such as via a graphical user interface (GUI). The automatic communication may or may not use direct contact. Some examples of automatic communication devices and methods include RFID, optical sensors, barcodes, NFC, and electrical connections. In some examples, the first valve 135 and the second valve 140 may be opened and closed in a predetermined pattern to adjust the amount of the airflow. The body 120 may also include seals for the apertures to prevent leakage of the scent medium 110 during shipping or storage, or while in use.

Further, the body 120 may have a single wall or a double wall. The body 120 and/or the scent medium 110 may be produced from a compostable or biodegradable material. In order to optimize the replaceable cartridge 105 for recycling, the body 120 and the scent medium 110 may be produced from the same material or from similar materials. Alternatively or additionally, the scent medium 110 may be derived from a non-petroleum based material.

The scent dispensing system 100 may be integrated into a vehicle. For example, the scent dispensing system 100 may be mounted on the dashboard, central armrest, roof, or headliner in a position that is visible to occupants of the vehicle. Alternatively, the scent dispensing system 100 may be arranged in a location that is not visible, not normally visible, or partially to occupants of the vehicle, such as in a glovebox, armrest compartment, door pocket, compartment with a window, etc. In these examples, the user may still observe the scent medium 110 when purchasing the replaceable cartridge 105 and/or when unpacking and/or installing the replaceable cartridge 105.

The scented air may be distributed directly into the cabin of the vehicle by the replaceable cartridge 105. In this case, the scent dispensing system 100 may be integrated into the heating, ventilation, and air conditioning (HVAC) system of the vehicle. Alternatively, the scented air may be distributed into the cabin via a delivery device, such as an airflow chamber or a duct. Alternatively, the scented air may be distributed indirectly into the cabin via ducting to HVAC system, or via ducting near the HVAC system. In other examples, the scent dispensing system 100 may be arranged in an area that may receive ambient or passive airflow. For example, the scent dispensing system 100 may be arranged inside or near a door, window, trunk, etc., such that when the door, window, or trunk is opened the scent is naturally released from the scent dispensing system 100.

Airflow into the replaceable cartridge 105 may be provided by the airflow source 145, which may include one or more fans. Alternatively, the airflow may be provided by ambient airflow and/or natural airflow, such as from external air. The airflow may be forced or induced by positive or negative pressure. For example, the airflow may be pushed and/or pulled.

The vehicle may include a user interface that is configured to display a visual representation of the scent medium 110. For example, the user interface may display a point cloud or an exploding sphere that is made up of points having the same shape and/or color as the scent medium 110. Accordingly, the user interface may provide sensory integration or congruence between the scent and the display. A multi-sensory experience, including components such as light, sound, temperature, haptics, etc., may be provided for the user.

The surface area of the scent medium 110 is configured to control the diffusion rate of the scent from the scent medium 110. The diffusion rate may be controlled via empirical and/or computational approaches. The surface area of the scent medium 110 affects how much scent is diffused into the cabin of the vehicle over a time period and how strong the scent is. As the surface area of the scent medium 110 increases, the diffusion rate increases. The airflow that is incident on the scent medium 110 also affects the diffusion rate. As the airflow increases, the diffusion rate increases. In some embodiments, if the airflow is large, the surface area of the scent medium 110 may be decreased. Alternatively, if the airflow is small, the surface area of the scent medium 110 may be increased. In some embodiments, the surface area of the scent medium 110 may be decreased in order to decrease the diffusion rate and increase the longevity of the replaceable cartridge 105.

As discussed in further detail below, the shape of the scent medium 110 may be selected to achieve the desired surface area and diffusion rate. In order to ensure a consistent diffusion rate, the shape of the scent medium 110 remains constant in different environmental conditions, such as high temperatures and/or pressures. The material into which the scent is injected to form the scent medium 110 is selected such that the scent medium 110 does not deform when subjected to different environmental conditions.

The shape of the scent medium 110 may also be selected to reflect characteristics of the scent. For example, the scent medium 110 may have an angular shape for a refreshing or sharp scent, such as mint or citrus. In other embodiments, the scent medium 110 may have a rounded shape for a warm scent, such as vanilla. In addition, the color of the scent medium 110 may be selected to correspond to the scent. For example, the scent medium 110 may be yellow for citrus or white for vanilla. Such correlations may provide sensory integration or congruence between the scent and the visual appearance of the scent medium 110.

The scent medium 110 may include a single scent carrier or a plurality of scent carriers. For example, the single scent carrier may have approximate major dimensions between 20 and 30 mm. A plurality of scent carriers may be arranged by various methods. For example, the chamber 115 may be randomly filled with a plurality of scent carriers. Alternatively, the plurality of scent carriers may be geometrically organized, stacked, interlocked, etc. A plurality of 10 to 20 scent carriers having interlocking shapes may each have approximate major dimensions between 5 and 10 mm. A plurality of 50 to 100 smaller scent carriers may each have approximate major dimensions between 3 and 6 mm. The scent medium 110 may include air cavities between adjacent ones of the plurality of scent carriers. The size of the air cavities may be adjusted to increase or decrease the airflow, which affects the amount of scent that is provided to the cabin of the vehicle. Increasing the size of the air cavities increases the airflow and the amount of scent that is provided to the cabin, while decreasing the size of the air cavities decreases the airflow and the amount of scent that is provided to the cabin.

As discussed above, the shape of the scent medium 110 may be selected to achieve the desired diffusion rate. For example, using a scent medium 110 having a hollow or open shape and/or a shape having an open cavity may increase the diffusion rate of the scent medium 110, due to the increased surface area of the scent medium 110. Some examples of these shapes include a toroid/donut, a tube with a round, oval, rectilinear, or polygonal cross-section that is open on both ends, and a cup that is open on one end.

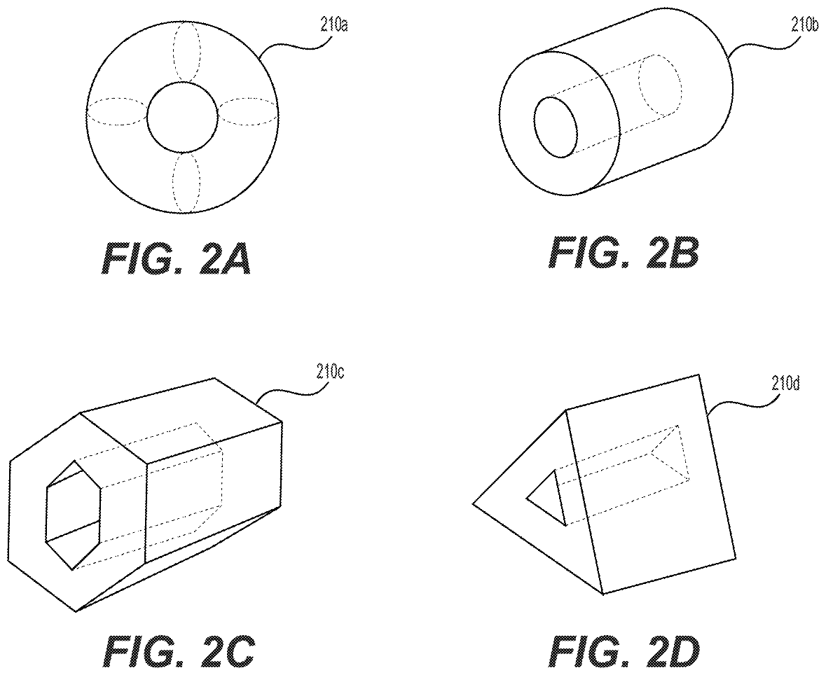

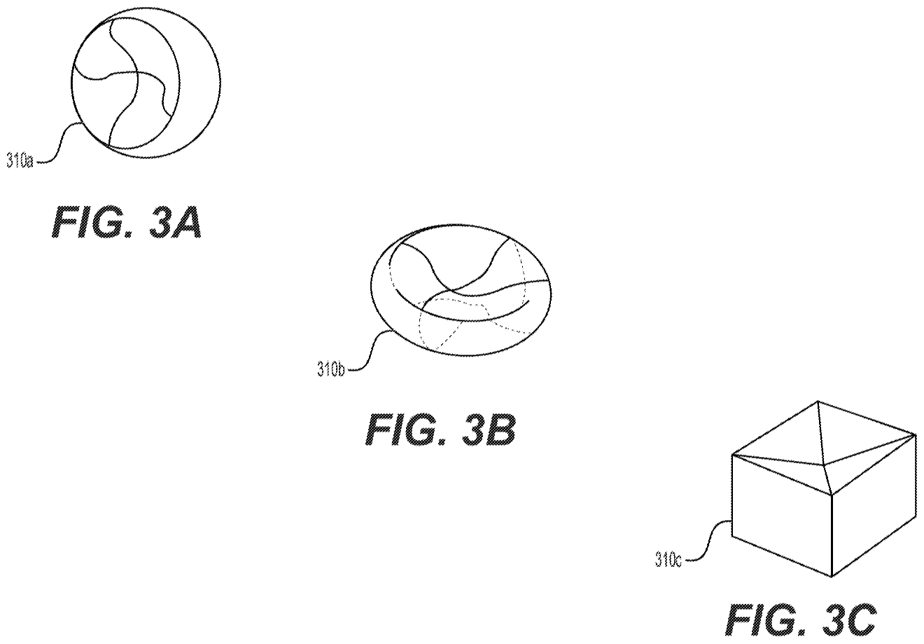

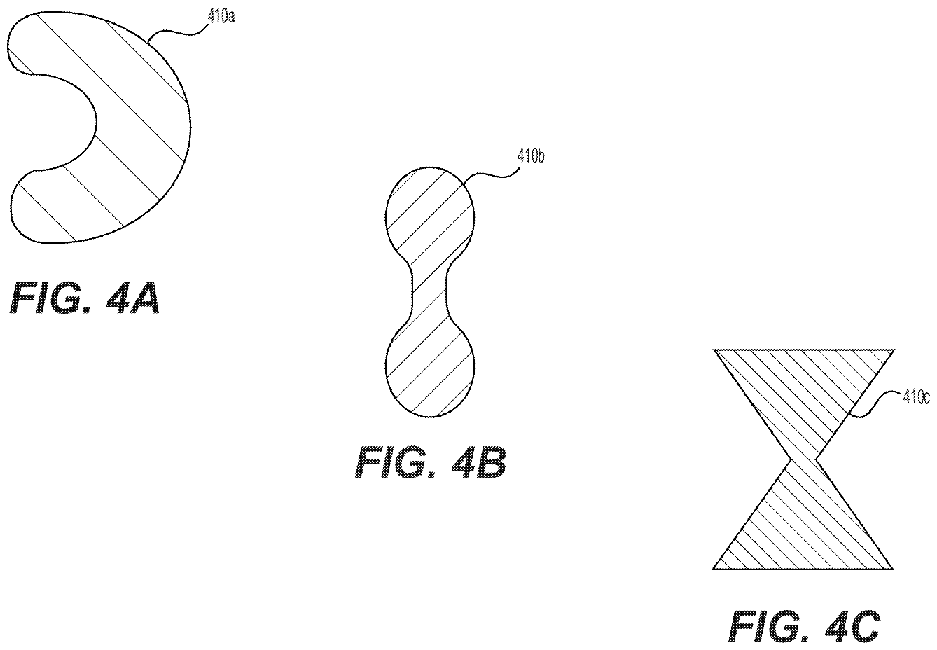

FIGS. 2A-2D show examples of scent carriers 210a-210d having hollow or open shapes. FIGS. 3A-3C show examples of scent carriers 310a-310c having open cavities, and FIGS. 4A-4C show the corresponding cross-sections 410a-410c of the scent carriers 310a-310c. The scent medium 110 may include one of the scent carriers 210a-210d or 310a-310c, or a plurality of the scent carriers 210a-210d or 310a-310c.

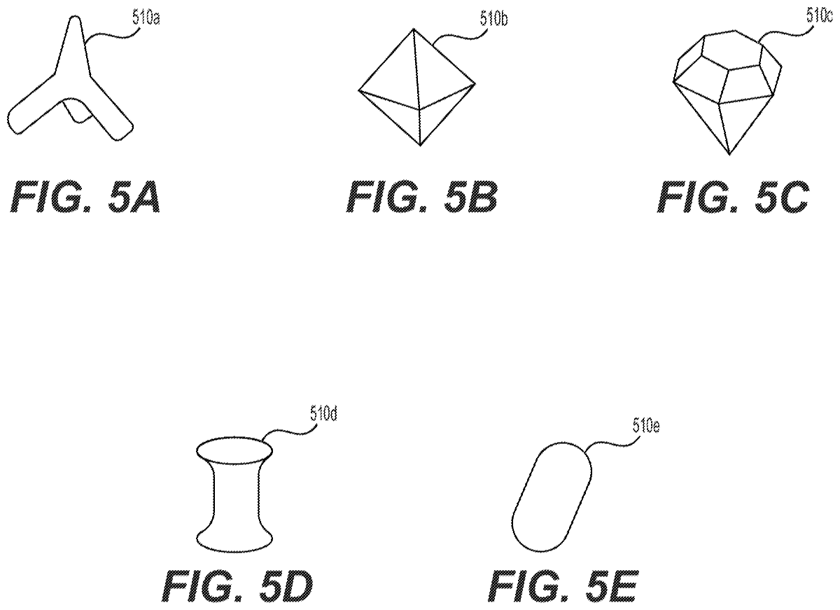

Alternatively, the scent medium 110 may have a simple geometric shape or a closed shape, such as a polyhedron, a tetrahedron, a Reuleaux triangle, or a caltrop. FIGS. 5A-5E show examples of scent carriers 510a-510e having closed shapes. The scent medium 110 may include one of the scent carriers 510a-510e, or a plurality of the scent carriers 510a-510e.

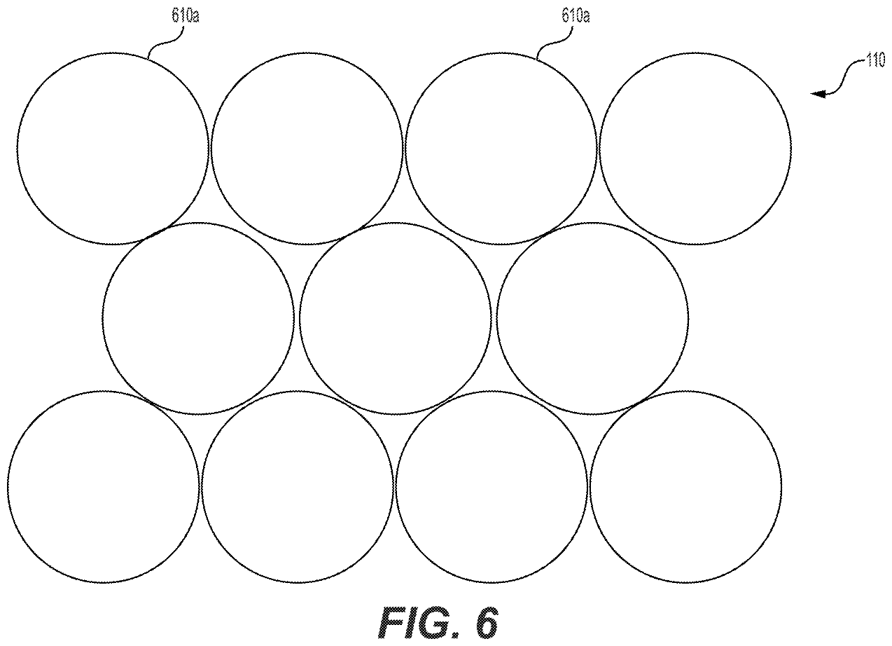

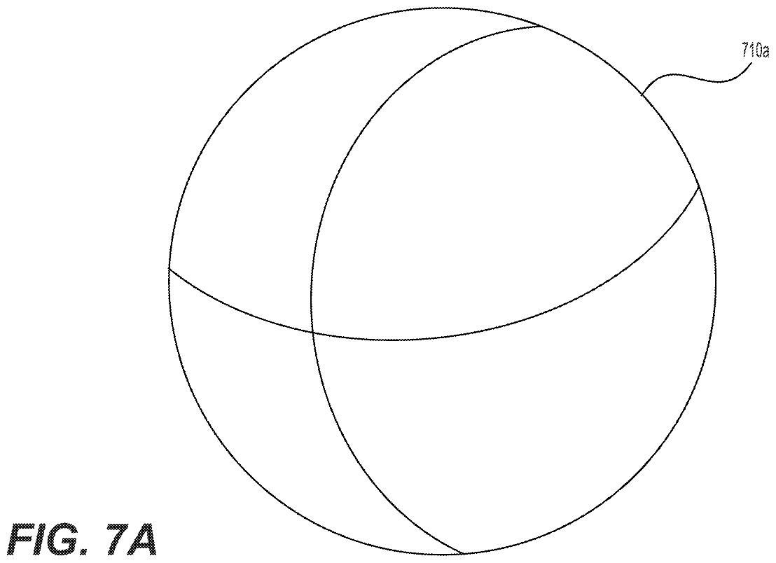

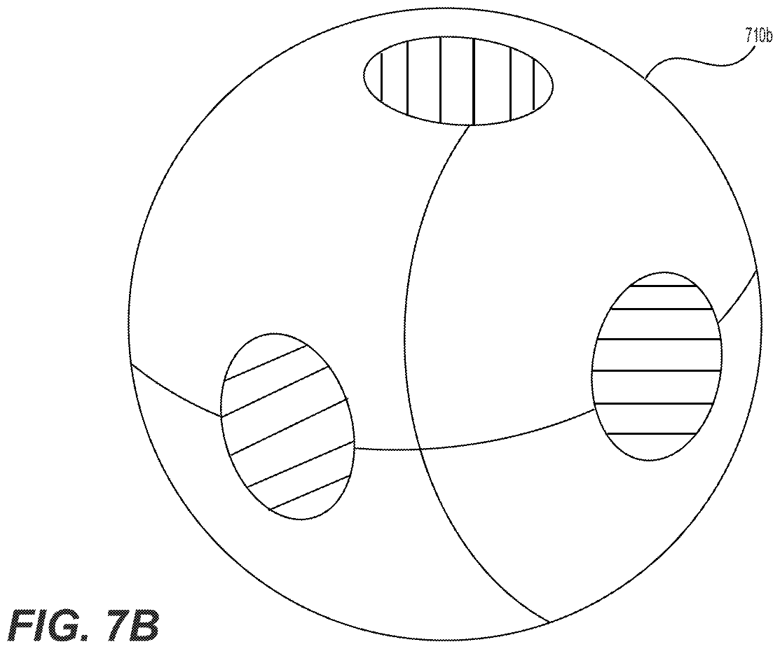

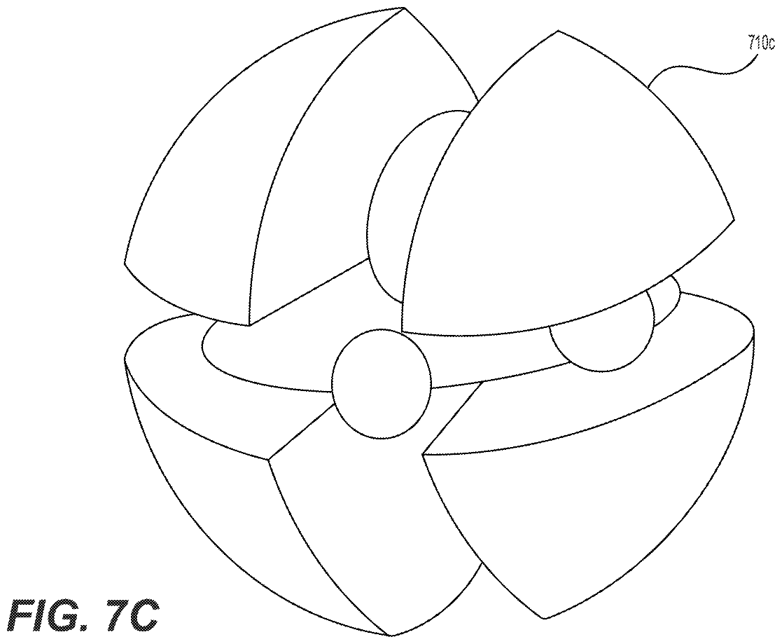

Alternatively, the scent medium 110 may include a plurality of self-organizing shapes. FIG. 6 shows an example in which the scent medium 110 includes a plurality of self-organizing scent carriers 610a. FIGS. 7A-7C show examples of scent carriers 710a-710c that may be used as the self-organizing scent carriers 610a. The scent carrier 710a is a solid shape, while the scent carrier 710b is a porous shape having one or more holes, and the scent carrier 710c is a complex shape having changes in its surface and solidity.

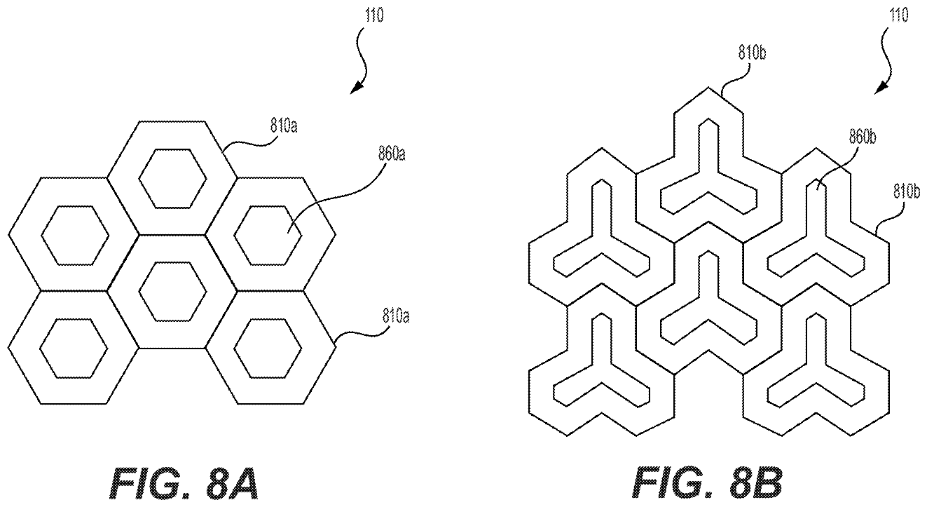

Alternatively, the scent medium 110 may include a plurality of interlocking scent carriers. FIG. 8A shows an example in which the scent medium 110 includes a plurality of interlocking scent carriers 810a, each of which includes an opening, aperture, or cavity 860a that may be formed as a closed through-hole. Similarly, FIG. 8B shows an example in which the scent medium 110 includes a plurality of interlocking scent carriers 810b, each of which includes an opening, aperture, or cavity 860b that may be formed as a closed through-hole.

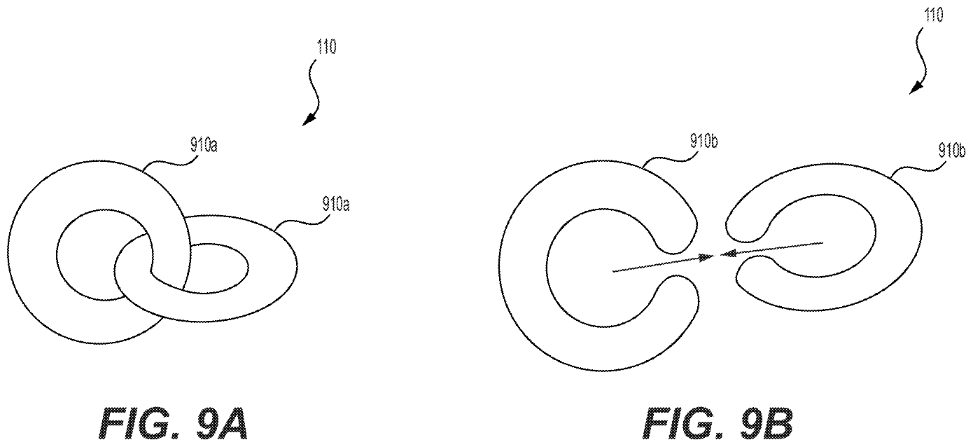

FIG. 9A shows another example in which the scent medium 110 includes a plurality of interlocking scent carriers 910a. The scent medium 110 shown in FIG. 9A may be produced by 3D printing. FIG. 9B shows yet another example in which the scent medium 110 includes a plurality of interlocking scent carriers 910b. The two open scent carriers 910b may interlock when moved towards each other and inserted together.

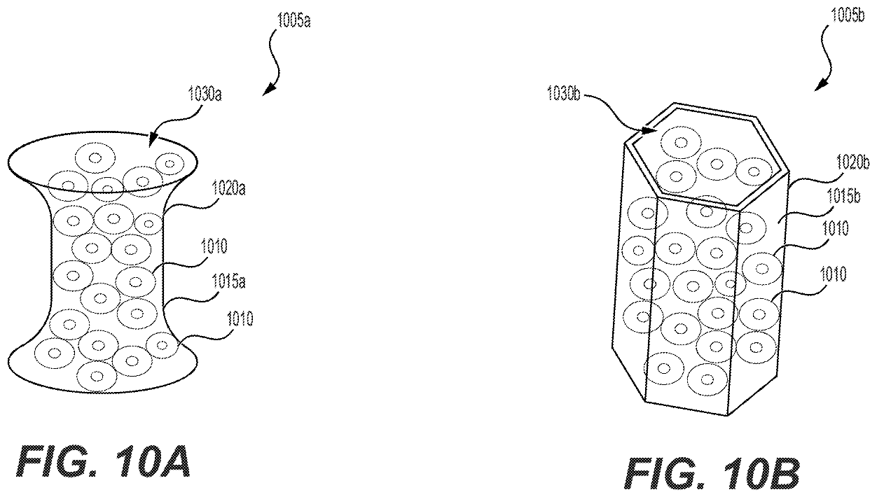

FIGS. 10A and 10B show examples of replaceable cartridges in which the scent medium includes a plurality of scent carriers. FIG. 10A shows an example of a replaceable cartridge 1005a that includes a body 1020a, an aperture 1030a, and a plurality of scent carriers 1010 within a chamber 1015a. Similarly, FIG. 10B shows an example of a replaceable cartridge 1005b that includes a body 1020b, an aperture 1030b, and a plurality of scent carriers 1010 within a chamber 1015b. In these examples, each of the plurality of scent carriers 1010 is formed as a toroid/donut.

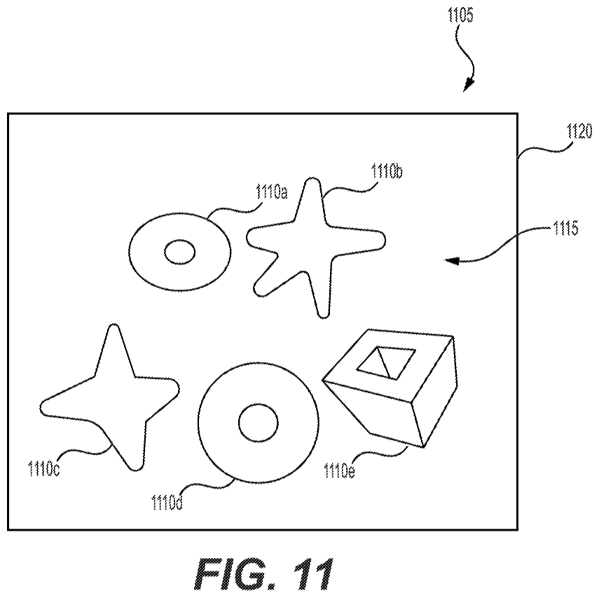

FIG. 11 shows an example of a replaceable cartridge in which the scent medium includes a plurality of scent carriers having different shapes. As shown in FIG. 11, the replaceable cartridge 1105 includes a body 1120 and a plurality of scent carriers 1110a-1110e having different shapes within a chamber 1115. More generally, the scent carriers 1110a-1110e may have different colors, textures, finishes, and/or shapes. This may be done to combine different scents or fragrance profiles into the body 1120. Further, this may allow the final fragrance profile to be customized depending on how the different scent carriers 1110a-1110e are combined in the body 1120. Multiple polymers may be used to create a unique appearance and to reflect the composition of a complex or sophisticated fragrance.

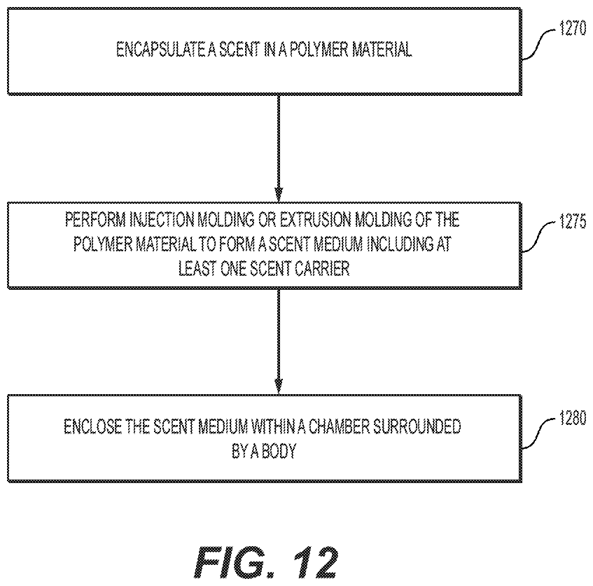

FIG. 12 shows a flowchart of a method of forming a replaceable cartridge for a scent dispensing system according to an exemplary embodiment of the invention. As shown in FIG. 12, the method 1200 begins by encapsulating a scent in a polymer material at block 1270. The polymer material may be polyethylene or a similar thermoplastic. In some examples, the polymer material may be a blend of polymers or include additives or pigments to control the properties of the material, such as visual or physical properties. For example, the polymer may include metallic, pearlescent, ceramic, and/or colored grains, specks, and/or elements. The scent is injected into the molten polymer, and the leadscrew of an injection molding or extrusion molding machine mixes the scent with the molten polymer.

The method 1200 continues by performing injection molding or extrusion molding of the polymer material to form a scent medium including at least one scent carrier. The forming tool of the injection molding or extrusion molding machine is used to form the at least one scent carrier to have at least one of the shapes discussed above. The shapes are formed to control the diffusion rate of the scent from the scent medium. Different materials and/or shapes may be co-extruded, co-injection molded, over-molded, twin-shot molded, or multi-shot molded.

The method 1200 continues by enclosing the scent medium within a chamber surrounded by a body. At least a portion of the body is transparent or translucent. The scent carriers may be integrally molded within the body or a portion of the body. The body is configured to display the scent medium to an observer.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

What is claimed is:

1. A replaceable cartridge for a scent dispensing system, the replaceable cartridge comprising:
a scent medium comprising at least one scent carrier, wherein:
a surface area of the scent medium is configured to control a diffusion rate of a scent from the scent medium, and
a shape of the scent medium remains constant in different environmental conditions; and
a body surrounding a chamber for housing the scent medium, wherein at least a portion of the body is transparent or translucent.

2. The replaceable cartridge according to claim 1, wherein the at least one scent carrier has a hollow shape or an open shape.

3. The replaceable cartridge according to claim 1, wherein the at least one scent carrier has an open cavity.

4. The replaceable cartridge according to claim 1, wherein the at least one scent carrier has a closed shape.

5. The replaceable cartridge according to claim 1, wherein:
the at least one scent carrier comprises a plurality of scent carriers, and
the scent medium further comprises air cavities between adjacent ones of the plurality of scent carriers.

6. The replaceable cartridge according to claim 1, wherein:
the at least one scent carrier comprises a plurality of scent carriers, and
some of the plurality of scent carriers have interlocking shapes.

7. The replaceable cartridge according to claim 1, wherein:
the at least one scent carrier comprises a plurality of scent carriers, and
respective surface areas of the plurality of scent carriers are configured to control the diffusion rate of the scent from the scent medium.

8. The replaceable cartridge according to claim 1, wherein at least one of a color, a texture, a gloss, or the shape of the scent medium is configured to correspond to the scent.

9. The replaceable cartridge according to claim 1, wherein at least one of the scent medium or the body comprises a compostable material.

10. The replaceable cartridge according to claim 1, wherein the body comprises an aperture that enables air to flow through the chamber.

11. The replaceable cartridge according to claim 10, further comprising a valve that is arranged at the aperture.

12. A scent dispensing system comprising:
the replaceable cartridge according to claim 11;
an airflow source that is configured to generate an airflow; and
a controller that is configured to control the airflow source and the valve to send the airflow through the chamber.

13. A vehicle comprising:
the scent dispensing system according to claim 12, wherein the scent dispensing system is integrated into a heating, ventilation, and air conditioning system of the vehicle; and
a user interface that is configured to display a visual representation of the scent medium.

14. A method of forming a replaceable cartridge for a scent dispensing system, the method comprising:
encapsulating a scent in a polymer material;
performing injection molding or extrusion molding of the polymer material to form a scent medium comprising at least one scent carrier, wherein:
a surface area of the scent medium is formed to control a diffusion rate of a scent from the scent medium, and
a shape of the scent medium is formed to remain constant in different environmental conditions; and
enclosing the scent medium within a chamber surrounded by a body, wherein at least a portion of the body is transparent or translucent.

15. The method according to claim 14, wherein the at least one scent carrier is formed to have a hollow shape or an open shape.

16. The method according to claim 14, wherein the at least one scent carrier is formed to have an open cavity.

17. The method according to claim 14, wherein the at least one scent carrier is formed to have a closed shape.

18. The method according to claim 14, wherein:
the at least one scent carrier comprises a plurality of scent carriers, and
the scent medium is formed to have air cavities between adjacent ones of the plurality of scent carriers.

19. The method according to claim 14, wherein:
the at least one scent carrier comprises a plurality of scent carriers, and
some of the plurality of scent carriers are formed to have interlocking shapes.

20. The method according to claim 14, wherein:
the at least one scent carrier comprises a plurality of scent carriers, and
respective surface areas of the plurality of scent carriers are formed to control the diffusion rate of the scent from the scent medium.
